(19) United States
(12) Patent Application Publication
(10) Pub. No.: US 2013/0026209 A1
(43) Pub. Date: Jan. 31, 2013

(54) Surgical Fastener Applying Apparatus

(71) Applicants: Patrick Mozdzierz, Glastonbury, CT (US); Christopher Penna, Guilford, CT (US)

(72) Inventors: Patrick Mozdzierz, Glastonbury, CT (US); Christopher Penna, Guilford, CT (US)

(21) Appl. No.: 13/191,717

(22) Filed: Jul. 27, 2011

(51) Int. Cl. A61B 17/068

(52) U.S. Cl. 227/180.1; 227/176.1

(57) ABSTRACT

A surgical stapler is provided. The surgical stapler includes a tubular body portion. A cartridge assembly (cartridge) is disposed at a distal end of the tubular body portion and defines a longitudinal axis therethrough. The cartridge includes a distal face having a stepped configuration defined by outer and inner steps arranged in descending order from the outer step to the inner step. An anvil member is disposed at the distal end of the tubular body portion and is positioned opposite the cartridge. The anvil member includes a proximal face having a stepped configuration defined by outer and inner steps arranged in descending order from the outer step to the inner step. The steps defined in the proximal face of the anvil member having a respective annular array of staple forming buckets disposed thereon to clinch the staples in tissue upon expulsion of the staples from the cartridge.

### BACKGROUND

1. Technical Field

The present disclosure relates to surgical stapling instruments for applying surgical fasteners or staples to body tissue and, more particularly to surgical stapling instruments for applying an annular array of surgical staples or fasteners.

2. Description of Related Art

Surgical stapling devices for applying an annular array of staples or fasteners to tissue are well known in the art. For example, such surgical stapling devices have particular utility in performing small bowl resection with end-to-end anastomosis. These devices, typically, include a staple pusher assembly or member and an anvil assembly or member at the distal end of the surgical stapling device. One or more annular or circular arrays of fasteners, such as, for example, staples, is operably housed in the cartridge assembly. The anvil member includes one or more corresponding annular arrays of staple forming bucket members that clinch or form (e.g., in a “B” staple formation) the staples after the staples are expelled from the cartridge assembly. In certain instances, each of the staples and staple forming buckets are arranged in the same plane on the respective cartridge assembly and anvil.

As is typical with conventional surgical stapling devices, the anvil member is movable from a retracted configuration (i.e., a spaced-apart from the cartridge assembly) for positioning tissue between the anvil member and the cartridge assembly, to an advanced (i.e., in close cooperative alignment with the staple cartridge) configuration for joining tissue, i.e., stapling the ends of a tubular organ in a body of the patient to be joined to create an anastomotic joint. A size of the anastomotic joint is, typically, limited to the size, e.g., diameter, of an annular array of formed staples that form the anastomotic joint the tissues.

After tissue is joined, a circular knife blade operably disposed within the cartridge assembly may be utilized to create a lumen through the joined tissue. However, because the knife blade is limited by geometry, i.e., a diameter of the knife blade is smaller than a diameter of the annular array of formed staples, the lumen created through the joined tissue is, typically, small in relation to the anvil member.

As can be appreciated, trying to remove the anvil through the relatively small lumen may prove to be arduous task. Moreover, the relatively small lumen or “stricture” may function as collection points for diviticular, which, in turn, may cause discomfort or pain to a patient or, worse, develop into a more serious ailment, e.g., diverticulitis, colon cancer, etc.

### SUMMARY

The present disclosure provides a surgical stapler. The surgical stapler includes a tubular body portion. A cartridge assembly is disposed at a distal end of the tubular body portion and defines a longitudinal axis therethrough. The cartridge assembly includes a distal face having a stepped configuration defined by outer and inner steps arranged in descending order from the outer step to the inner step. The outer and inner steps lying in respective outer and inner planes that are disposed parallel to one another and perpendicular to the longitudinal axis defined though the cartridge assembly. The outer and inner steps defined in the distal face of the cartridge assembly having a respective annular array of outer and inner slots defined therethrough and configured to house a respective outer and inner annular array of staples. An anvil member is disposed at the distal end of the tubular body portion and is positioned opposite the cartridge assembly. The anvil member includes a proximal face having a stepped configuration defined by outer and inner steps arranged in descending order from the outer step to the inner step. The outer and inner steps defined in the proximal face of the anvil member having a respective annular array of staple forming buckets disposed thereon to clinch the staples in tissue upon expulsion of the staples from the cartridge assembly such that clinched tissue forms a generally funnel shape.

In certain embodiments, the cartridge assembly further includes a middle step lying in a middle plane that is disposed parallel to inner and outer planes and perpendicular to the longitudinal axis defined though the cartridge assembly. The middle step is defined in the distal face of the cartridge assembly and has a respective annular array of middle slots defined therethrough. The middle step is configured to house respective middle annular array of staples. In this embodiment, the anvil member further includes a middle step disposed between the outer and inner steps and has an annular array of staple forming buckets disposed thereon to clinch the corresponding staples disposed in the middle step of the cartridge assembly.

The present disclosure provides a surgical stapler. The surgical stapler includes a tubular body portion. A cartridge assembly is disposed at a distal end of the tubular body portion and defines a longitudinal axis therethrough. The cartridge assembly includes a distal face having a stepped configuration defined by a plurality of steps including a first uninterrupted step lying in a first plane and a second uninterrupted step lying in a second plane. The first and second planes of the first and second steps of the distal face being perpendicular to the longitudinal axis defined through the cartridge assembly and parallel to one another. The first step having an annular array of first slots defined therethrough and the second step having an annular array of second slots defined therethrough. The first and second annular arrays of slots configured to house a corresponding first and second annular array of staples. An anvil member is disposed at the distal end of the tubular body portion and positioned opposite the cartridge assembly. The anvil member includes a proximal face having a stepped configuration with an annular array of staple forming buckets disposed thereon to clinch the staples in tissue upon expulsion of the staples from the cartridge assembly such that clinched tissue forms a generally funnel shape.

The present disclosure also provides a staple fastening assembly. The staple fastening assembly including a cartridge assembly adapted to connect to a surgical stapler and defining a first longitudinal axis therethrough. The cartridge assembly includes a distal face having a stepped configuration defined by a plurality of steps including a first uninterrupted step lying in a first plane and a second uninterrupted step lying in a second plane. The first and second planes of the first and second steps of the distal face being perpendicular to the longitudinal axis defined through the cartridge assembly and parallel to one another. The first step having an annular array of first slots defined therethrough and the second step having an annular array of second slots defined therethrough. The first and second annular arrays of slots configured to house a corresponding first and second annular array of staples. An anvil member is disposed at the distal end of the tubular body portion and positioned opposite the cartridge assembly. The anvil member includes a proximal face having a stepped configuration with an annular array of staple forming buckets disposed thereon to clinch the staples in tissue upon expulsion of the staples from the cartridge assembly such that clinched tissue forms a generally funnel shape.

### BRIEF DESCRIPTION OF THE DRAWING

Various embodiments of the present disclosure are described hereinbelow with references to the drawings, wherein:

FIG. 1 is a perspective view of a surgical stapling apparatus including a staple fastening assembly including an anvil member and a cartridge assembly according to an embodiment of the present disclosure;

FIG. 2 is a perspective view of an enlarged area of detail depicted in FIG. 1;

FIG. 3 is an exploded view of the cartridge assembly depicted in FIG. 1 with the parts separated;

FIG. 4 is a partial, cross-sectional view of the cartridge assembly depicted in FIG. 3;

FIG. 5 is a partial, cross-sectional view of an anvil depicted in FIG. 1;

FIG. 6 is a cross-sectional view of the anvil and cartridge assembly coupled to one another;

FIG. 7 is a perspective view of an enlarged area of detail depicted in FIG. 6;

FIG. 8 is a partial, cross-sectional view of the anvil and cartridge assembly in a partially approximated configuration;

FIG. 9 is a side view of the anvil and cartridge assembly approximated toward one another and with tissue positioned therebetween;

FIG. 10 is a side view of tissue that has been stapled utilizing a conventional surgical stapling device; and

FIG. 11 is a side view of tissue that has been stapled utilizing the surgical stapling device depicted in FIG. 1.

### DETAILED DESCRIPTION

Detailed embodiments of the present disclosure are disclosed herein; however, the disclosed embodiments are merely examples of the disclosure, which may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure.

In the drawings and in the descriptions that follow, the term “proximal,” as is traditional, will refer to the end of a surgical instrument that is closer to the user, while the term “distal” will refer to the end of the surgical instrument that is farther from the user.

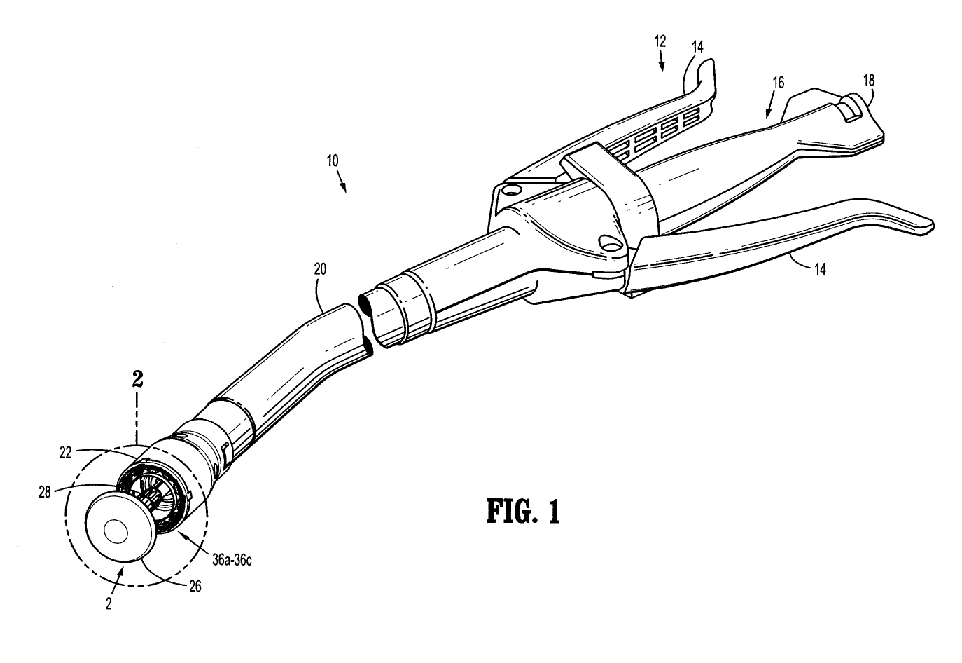

Referring now in specific detail to the drawings, in which like reference numerals identify similar or identical elements throughout the several views, FIG. 1 shows a staple fastening assembly 2 (assembly 2) adapted for use with a surgical stapling apparatus 10 (apparatus 10) in accordance with an embodiment of the present disclosure. Apparatus 10 is configured to perform a circular anastomosis of a tubular organ. Briefly, apparatus 10 includes a handle assembly 12 having one or more pivotable actuating handle members 14 (two pivotable handle members 14 shown in the drawings). Apparatus 10 includes an advancing device 16 including a rotatable grip member 18 that is configured to approximate an anvil member 26 towards a cartridge assembly 22. Extending from handle assembly 12 is a tubular body portion 20 that includes a generally curved configuration. In certain embodiments, body portion 20 may also be straight and, in other embodiments, may be flexible to bend to any configuration. Body portion 20 terminates in assembly 2 including cartridge assembly 22 that is associated with an annular array of staples 36a-36c, see FIGS. 1-3. Anvil member 26 is positioned opposite cartridge assembly 22 and is connected to apparatus 10 by shaft 28 at connection device or structure (not explicitly shown) operably disposed within the cartridge assembly 22. For a more detailed description of the body portion 20, advancing device 16, handle assembly 12 including handle members 14 reference is made to commonly-owned U.S. Pat. No. 5,915,616 to Viola et al., filed on Oct. 10, 1997, which is incorporated in its entirety herein by reference.

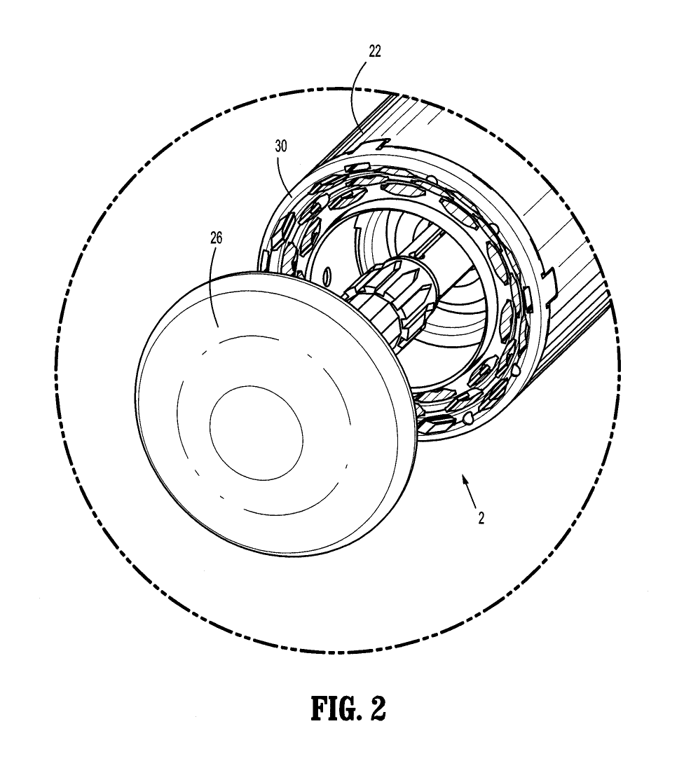

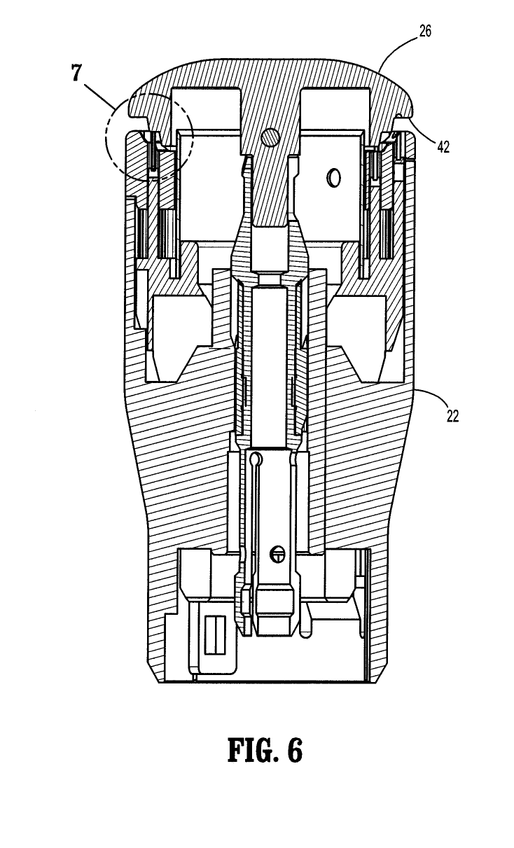

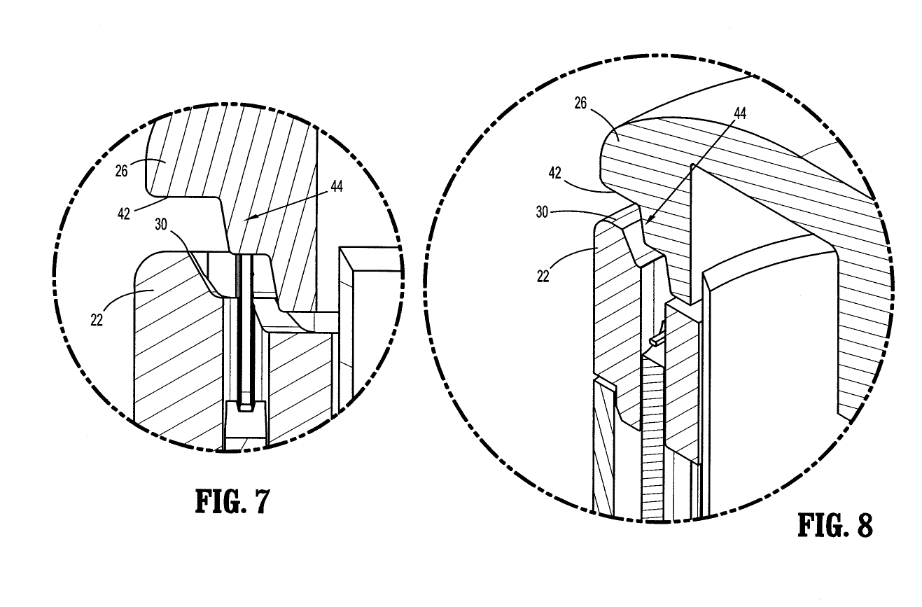

With reference to now to FIGS. 2-9, assembly 2 including the anvil member 26 and cartridge assembly 22 according to an embodiment of the present disclosure is illustrated. In accordance with the present disclosure, each of the anvil 26 and cartridge 22 includes staggered levels of planes that are configured to contact tissue “T” and shape the tissue “T” subsequent to stapling or clinching the tissue “T” such that the stapled or clinched tissue “T” forms a tapered or funnel shape, see FIG. 11 for example.

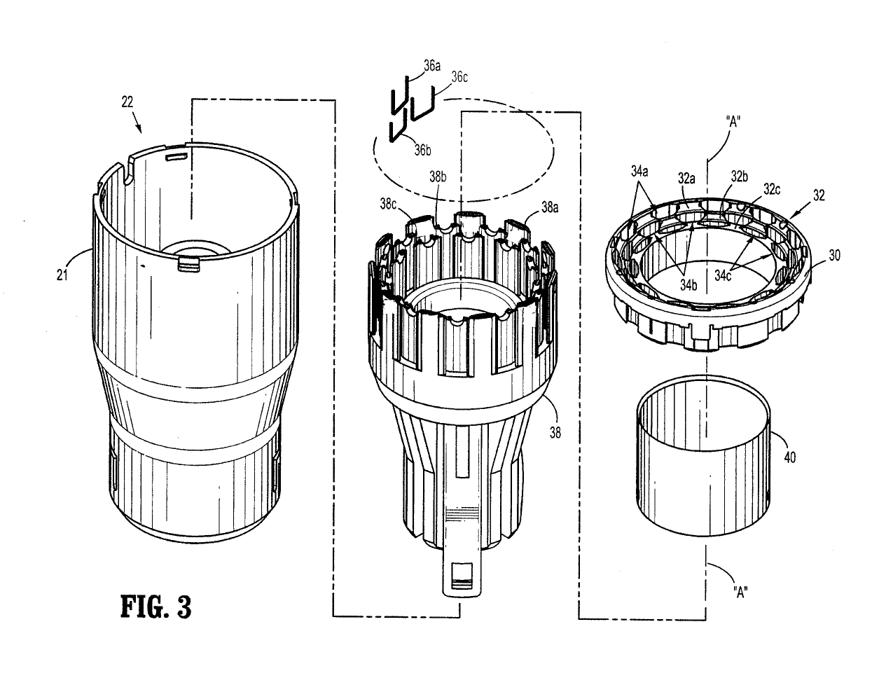

Continuing with reference to FIGS. 2 and 3, cartridge assembly 22 (cartridge 22) defines a longitudinal axis “A-A” therethrough. A housing 21 (FIG. 3) of suitable configuration is configured to house and/or support a staple pusher member 38 and a distal face 30 of the cartridge 22.

Distal face 30 has a stepped configuration including two or more steps 32 (FIGS. 2-4). In the illustrated embodiment, the distal face 30 includes three (3) steps 32 defined by an outer step 32a (step 32a), a middle step 32b (step 32b) and an inner step 32c (step 32c), see FIGS. 2-4. Each of the steps 32a-32c includes a generally flat or “uninterrupted” (or “continuous”) configuration. This “uninterrupted” configuration allows for a maximum amount of staples to be expelled from the cartridge 22 for a given surface area of the steps 32a-32c. As can be appreciated, this may facilitate in achieving various degrees of staple-line formations, i.e., the “tightness” of a given staple-line formation for a given step.

Formed on the distal face 30 during the manufacturing process thereof, steps 32a-32c are arranged in descending order from the step 32a to the step 32c. That is, step 32a (the outer step) is disposed distal of the step 32b that is disposed distal of the step 32c (the inner step). Steps 32a-32c lie in respective outer, middle and inner planes that are disposed parallel to one another and perpendicular to the longitudinal axis “A-A” defined though the cartridge 22, as best seen in FIG. 3.

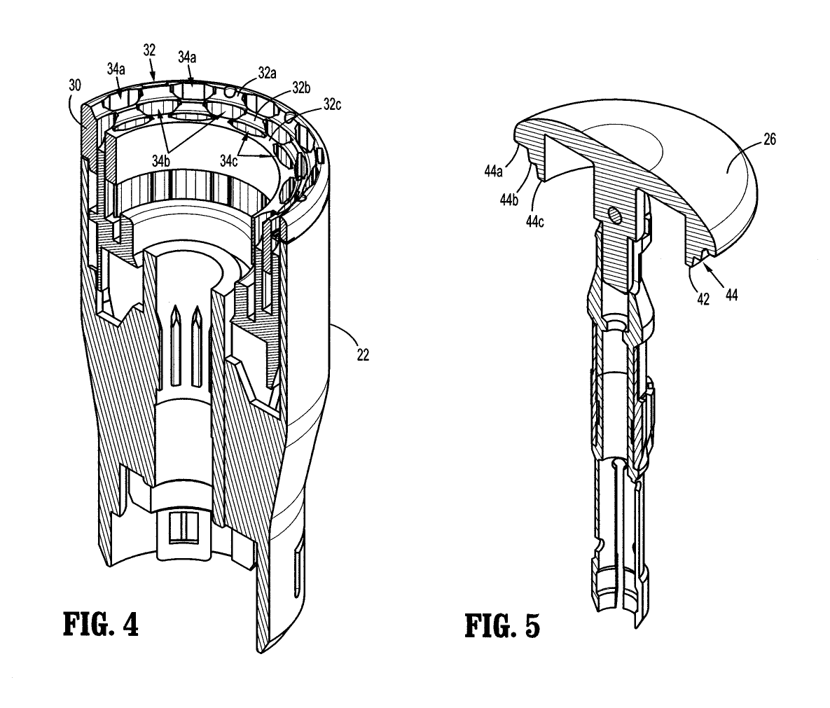

Respective annular arrays of outer, middle and inner slots 34a-34c are defined through respective steps 32a-32c and are configured to house respective outer, middle and inner annular array of staples 36a-36c, see FIGS. 3 and 4. For illustrative purposes, one of each of the staples 36a-36c is shown in the representative figures. To provide a “tight” staple line formation, the inner and outer slots 34a and 34c, and, thus, the staples 36a-36c are radially aligned with each other and the middle slots 34b are radially disposed therebetween, see FIGS. 3 and 4. However, other configurations of the slots 34a-34c are contemplated to provide various staple line formations, such as, for example, to accommodate specific surgical procedures, specific contemplated manufacturer uses, etc.

Slots 34a-34c are configured to maintain the staples 36a-36c in a substantially fixed orientation on corresponding staple pusher members 38a-38c formed on staple pusher member 38, see FIG. 1 in combination with FIG. 3. Staples 34a-34c may include any suitable length size. For example, and in one particular embodiment, the length of the staples 34a-34c ranges from about 3.0 mm to about 5.0 mm.

Staple pusher member 38 includes a stepped configuration defined by outer, middle and inner annular arrays of pushers 38a-38c. Pushers 38a-38c are aligned with respective slots 34a-34c for expelling the staples 36a-36c therefrom and towards anvil member 26.

A circular knife 40 (or other suitable device) is disposed within the cartridge 22 and operable to sever tissue “T” subsequent to tissue “T” being clinched (FIG. 3). In particular, the knife 40 is operably coupled to the inner row of staple pushers 38c and follows the application of the staples 36a-36c to excise unwanted tissue at the anastomosis site.

With reference to FIGS. 1, 2 and 5-9 anvil member 26 is disposed at the distal end of the tubular body portion 20 and positioned opposite the cartridge 22. The anvil member 26 includes a proximal face 42 having a corresponding stepped configuration defined by one or more steps 44. In the illustrated embodiment, steps 44 includes outer, middle and inner steps 44a-44c arranged in descending order from the outer step 44a to the inner step 44c. That is, step 44a (the outer step) is disposed distal of the step 44b that is disposed distal of the step 44c (the inner step). In the illustrated embodiment, steps 44a-44c of the anvil member 26 has respective annular arrays of staple forming buckets (not explicitly shown) disposed thereon to clinch the staples 36a-36c in tissue “T” upon expulsion of the staples 36a-36c from the cartridge 22 such that clinched tissue forms a generally funnel shape, as best seen in FIG. 11.

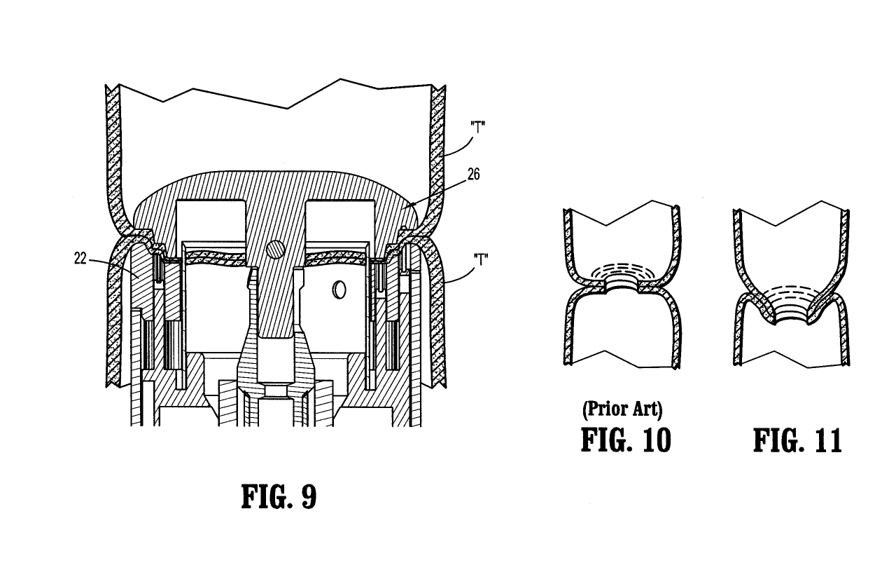

In use, tissue “T” is positioned between the anvil 26 and cartridge 22. The anvil 26 is approximated towards the cartridge 22 (FIG. 9). Thereafter, staples 36a-36c are expelled from the cartridge 22 towards the corresponding staple forming buckets to clinch or staple tissue “T.” Subsequently, knife 40 may be utilized to sever tissue to create an opening or lumen therethrough. The clinched or stapled tissue “T” has a generally tapered or funnel shape (FIG. 11) as compared to tissue clinched or stapled utilizing a conventional stapler, see FIG. 10 for example. The unique configuration of the steps 44a-44c of the anvil 26 and the steps 32a-32c including respective slots 34a-34 that communicate with staple pushers 38a-38c provides a staple-line formation that overcomes the aforementioned drawbacks, typically, associated with conventional surgical staplers. That is, the tapered or funnel shape of the clinched or stapled tissue “T” enables larger openings or lumens to be attained in the end-to-end anastomosis while maintaining a superior surface area of the anastomotic joint. Moreover, the geometry of the anastomosis enables an easy removal of the anvil 26 through the clinched or stapled tissue “T” and potential reduction of “stricture” and diviticular collection points.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A surgical stapler, comprising:
a tubular body portion;
a cartridge assembly disposed at a distal end of the tubular body portion and defining a longitudinal axis therethrough, the cartridge assembly including a distal face having a stepped configuration defined by outer and inner steps arranged in descending order from the outer step to the inner step, the outer and inner steps lying in respective outer and inner planes that are disposed parallel to one another and perpendicular to the longitudinal axis defined though the cartridge assembly, the outer and inner steps defined in the distal face of the cartridge assembly having respective annular arrays of outer and inner slots defined therethrough and being configured to house respective outer and inner annular arrays of staples; and
an anvil member disposed at the distal end of the tubular body portion and positioned opposite the cartridge assembly, the anvil member including a proximal face having a corresponding stepped configuration defined by outer and inner steps arranged in descending order from the outer step to the inner step, the outer and inner steps defined in the proximal face of the anvil member having respective annular arrays of staple forming buckets disposed thereon to clinch the staples in tissue upon expulsion of the staples from the cartridge assembly such that clinched tissue forms a generally funnel shape.

2. A surgical stapler according to claim 1, further comprising:
wherein the cartridge assembly further includes a middle step lying in a middle plane that is disposed parallel to inner and outer planes and perpendicular to the longitudinal axis defined though the cartridge assembly, the middle step defined in the distal face of the cartridge assembly and having respective an annular array of middle slots defined therethrough and configured to house respective middle annular array of staples; and
wherein the anvil member further includes a middle step disposed between the outer and inner steps and has an annular array of staple forming buckets disposed thereon to clinch the corresponding staples disposed in the middle step of the cartridge assembly in tissue upon expulsion of the staples from the cartridge assembly such that clinched tissue forms a generally funnel shape.

3. A surgical stapler according to claim 2, wherein the inner and outer slots are radially aligned with each other and the middle slots are radially disposed therebetween.

4. A surgical stapler according to claim 1, wherein a staple pusher member is in operative communication with the cartridge assembly for expelling the staples, the staple pusher member including a stepped configuration defined by outer and inner annular arrays of pushers corresponding to the outer and inner annular arrays of slots and staples.

5. A surgical stapler according to claim 2, wherein the outer and inner staple forming buckets are radially aligned with each other and the middle staple forming buckets are radially disposed therebetween.

6. A surgical stapler according to claim 1, wherein a knife is operably disposed within the cartridge assembly and operable to sever tissue subsequent to tissue being clinched.

7. A surgical stapler according to claim 5, wherein the surgical stapler further includes an actuation member configured to move the knife towards the anvil member.

8. A surgical stapler, comprising:
a tubular body portion;
a cartridge assembly disposed at a distal end of the tubular body portion and defining a longitudinal axis therethrough, the cartridge assembly including a distal face having a stepped configuration defined by a plurality of steps including a first uninterrupted step lying in a first plane and a second uninterrupted step lying in a second plane, the first and second planes of the first and second steps of the distal face being perpendicular to the longitudinal axis defined through the cartridge assembly and parallel to one another, the first step having an annular array of first slots defined therethrough and the second step having an annular array of second slots defined therethrough, the first and second annular arrays of slots configured to house a corresponding first and second annular array of staples; and
an anvil member disposed at the distal end of the tubular body portion and positioned opposite the cartridge assembly, the anvil member including a proximal face having a stepped configuration with an annular array of staple forming buckets disposed thereon to clinch the staples in tissue upon expulsion of the staples from the cartridge assembly such that clinched tissue forms a generally funnel shape.

9. A surgical stapler according to claim 8, wherein the distal face further includes a third uninterrupted step lying in a third plane disposed perpendicular to the longitudinal axis defined through the cartridge assembly and parallel to the first and second planes of the first and second steps of the distal face of the cartridge assembly, the third step having an annular array of third slots defined therethrough and configured to house a corresponding third annular array of staples.

10. A surgical stapler according to claim 9, wherein the first and third slots are radially aligned with each other and the second slots are radially disposed therebetween.

11. A surgical stapler according to claim 9, wherein a staple pusher member is in operative communication with the cartridge assembly for expelling the staples, the staple pusher member including a stepped configuration defined by first, second and third annular arrays of pushers corresponding to the first, second and third annular arrays of slots and staples.

12. A surgical stapler according to claim 8, wherein the stepped configuration of the proximal face of the anvil member includes a corresponding first uninterrupted step lying in a first plane and a corresponding second uninterrupted step lying in a second plane, the first and second planes of the first and second steps of the proximal face disposed perpendicular to the longitudinal axis of the cartridge assembly and parallel to one another.

13. A surgical stapler according to claim 12, wherein an annular array of first staple forming buckets is defined through the first step of the anvil member and an annular array of second staple forming buckets is defined through the second step of the anvil member.

14. A surgical stapler according to claim 13, wherein the anvil member further includes a third uninterrupted step lying in a third plane of the proximal face, the third plane of third uninterrupted step being perpendicular to the longitudinal axis defined through the cartridge assembly and parallel to the first and second planes of the first and second steps of the proximal face of the anvil member, the third step having an annular array of third staple forming buckets defined therethrough.

15. A surgical stapler according to claim 14, wherein the first and third staple forming buckets are radially aligned with each other and the second staple forming buckets are radially disposed therebetween.

16. A surgical stapler according to claim 8, wherein a knife is operably disposed within the cartridge assembly and operable to sever tissue subsequent to tissue being clinched.

17. A surgical stapler according to claim 16, wherein the surgical stapler further includes an actuation member configured to move the knife towards the anvil member.

18. A staple fastening assembly, comprising:
a cartridge assembly adapted to connect to a surgical stapler and defining a first longitudinal axis therethrough, the cartridge assembly member including a distal face having a stepped configuration defined by outer, middle and inner steps arranged in descending order from the outer step to the inner step, the outer, middle and inner steps lying in respective outer, middle and inner planes that are disposed parallel to one another and perpendicular to the first longitudinal axis defined though the cartridge assembly, the outer, middle and inner steps defined in the distal face of the cartridge assembly having a respective annular array of outer, middle and inner slots defined therethrough and configured to house a respective outer, middle and inner annular array of staples; and
an anvil member disposed at the distal end of the tubular body portion and positioned opposite the cartridge assembly, the anvil member including a proximal face having a corresponding stepped configuration defined by outer, middle and inner steps arranged in descending order from the outer step to the inner step, the outer, middle and inner steps defined in the proximal face of the anvil member having a respective annular array of staple forming buckets disposed thereon to clinch the staples in tissue upon expulsion of the staples from the cartridge assembly such that clinched tissue forms a generally funnel shape.

19. A staple fastening assembly according to claim 18, wherein the inner and outer slots are radially aligned with each other and the middle slots are radially disposed therebetween and the outer and inner staple forming buckets are radially aligned with each other and the middle staple forming buckets are radially disposed therebetween.

20. A staple fastening assembly according to claim 19, wherein a staple pusher member of the cartridge assembly is configured for expelling the staples from the cartridge assembly, the staple pusher member including a stepped configuration defined by outer, middle and inner annular arrays of pushers corresponding to the outer, middle and inner annular arrays of slots and staples.

21. A staple fastening assembly according to claim 18, wherein the staple fastening assembly is configured to releasably couple to a distal end of the surgical stapler, the surgical stapler comprising:
a knife that operable to sever tissue subsequent to tissue being clinched; and
an actuation member configured to move the knife towards the anvil member.
